(19) United States
(12) Patent Application Publication
(10) Pub. No.: US 2002/0010945 A1
(43) Pub. Date: Jan. 24, 2002

(54) Kelch family of proteins and use thereof

(76) Inventor: Michael B. McKeown, Barrington, RI (US)

Correspondence Address: Stephen E. Reiter, FOLEY & LARDNER, 23rd Floor, 402 West Broadway, San Diego, CA, 92101-3542, US

(21) Appl. No.: 09/780,258

(22) Filed: Feb. 9, 2001

Related U.S. Application Data
(60) Provisional application No. 60/181,736, filed on Feb. 11, 2000.

(51) Int. Cl. A01K 67/00; A01K 67/033; C12N 9/64; A61K 48/00

(52) U.S. Cl. 800/8; 435/226; 514/44; 514/12

(57) ABSTRACT

The present invention relates generally to factors contributing to neurodegenerative behavior, and more specifically to the Kelch family of genes. It has been identified that mutant forms of Kelch lead to significant deterioration of behavior and body control while not decreasing life span, a phenotype similar to many human deterioration diseases. The present invention establishes that the Kelch family of genes is involved in maintenance of proper neural function with aging, e.g., in animals, including insects and mammal (including humans).

### FIELD OF THE INVENTION

The present invention relates generally to factors contributing to neurodegenerative behavior, and more specifically to the Kelch family of genes. It has been discovered that mutant forms of Kelch lead to significant deterioration of behavior and body control while not decreasing life span, a phenotype similar to many human deterioration diseases. The present invention establishes that the Kelch family of genes is involved in maintenance of proper neural function with aging in animals, e.g., in insects and mammal (including humans).

### BACKGROUND OF THE INVENTION

Even “normal” aging results in declines in cognitive and other neural performance. Age dependent neural degenerative diseases lead to more significant and rapid debility. Alzheimer's Disease and cerebrovascular disease are the most common causes of dementia, but various other causes account for dementia in a significant fraction of patients (Tatemichi, et al. (1994) Alzheimer Disease. Terry, Katzman and Bick ed. Raven Press. New York. 123-166, incorporated by reference herein), often in the context of a near normal life span. Although substantial progress has been made in characterizing the phenotypes of various neurodegenerative diseases and in identifying mutant genes contributing to such diseases, much is yet to be learned. For example, three autosomal dominant mutations (in the genes for the Amyloid Precursor Protein, presenilin-1 and presenilin-2) have been identified as causing approximately 5% of the cases of Alzheimer's Disease, and the APOE epsilon 4 variant is a major risk factor which appears to contribute to 20% of all Alzheimer cases (see Cruts, M. and Van Broeckhoven, C. (1998) Ann Med. 30:560-565, incorporated by reference herein). Thus, key genes are known, but the genes and environmental factors contributing to a major fraction of Alzheimer's Disease are yet to be discovered.

Many have recognized that identifying a gene that contributes to a disease serves as a wedge to open the disease to more focused molecular and genetic study, even when the majority of cases are sporadic or appear polygenic. This realization has lead researchers to adopt genetic approaches to study neurodegeneration in various animals, including insects such as Drosophila. Drosophila has been widely used in genetic analysis because it offers the advantages of being genetically tractable and amenable to behavioral analysis. Sufficient data are now available to show that the structural and functional similarity of Drosophila and human genes controlling key aspects of development extends to genes involved in neural functions and survival (Mutsuddi, M. and Nambu, J. R. (1998) Curr Biol. 8:R-809-R811, incorporated by reference herein). A summary of Drosophila genes which display neural degenerative phenotypes is presented in Table 1.

TABLE 1
Drosophila Genes with Neural Degeneration Phenotypes

| | Human Homolog or |
|---|---|
| | Phenotype of Novel |
| Drosophila Gene | Mutations |
| Genes with Identified | Human Homolog or Disease |
| Human Homologs | beta-amyloid precursor |
| Beta-amyloid protein | presenilin 1 and 2 |
| Presenilin | superoxide dismutase |
| Superoxide dismutase | degenerin sodium channel |
| Ripped pocket/ | neuropathy target esterase |
| pickpocket | Increase in very long chain fatty acids, |
| Swiss cheese | rescued with “Lorenzo's oil” |
| Bubblegum | Phenotype |
| | Glial hypo-wrapping |
| Genes without | Axon degeneration and fusion |
| Identified Human | Neuronal and glial degeneration, |
| Homologs | multilamellar structures |
| Drop-dead | Optic lobe degeneration |
| Spongecake | circling behavior and brain degeneration |
| Eggroll | |
| Vacuolar medulla | |
| pirouette | |

There are a number of notable features apparent from Table 1. The first is the high degree of similarity between Drosophila proteins and proteins associated with aging or neural disorders in humans. Consider the Swiss Cheese protein (Kretzschmar, et al. (1997) J Neurosci. 17:7425-7432, incorporated by reference herein), which BLAST scores as having 44% identity to the human neuropathy target esterase, including one region of 266 amino acids with >65% identity (Lush, et al. (1998) Biochem J. 332:1-4, incorporated by reference herein). Furthermore, the similarity between Drosophila and human extends to function. For example, the Drosophila Appl (amyloid protein gene) loss-of-function mutations are rescued equally by wild type Drosophila and human genes (Luo, et al. (1992) Neuron. 9:595-605, incorporated by reference herein).

A second striking point about Table 1 is the array of phenotypes observed for Drosophila mutants, and the similarity to various human disease phenotypes. For example, spongecake mutant brains have defects resembling those seen in brains from patients with spongiform degenerations while eggroll mutant brains show multilamellated structures similar to those seen in Tay-Sachs (Min, K. T. and Benzer, S. (1997) Curr Biol. 7:885-888, incorporated by reference herein). This phenotypic similarity also occurs with expression of mutant human proteins in Drosophila. For example, the expanded polyglutamine regions of the spinocerebellar ataxia type 3 protein or of the Huntington's disease protein induced neural degeneration in Drosophila (Jackson, et al. (1998) Neuron. 21:633-642.; Warrick, et al. (1998) Cell. 93:939-949, each herein incorporated by reference).

A third point from Table 1 is that Drosophila mutants can predict proteins involved in human disease. For example, Swiss cheese was first identified based on defects in adult brain morphology in Drosophila. No mammalian homolog was identified when the gene was first cloned (Kretzschmar et al. (1997)). Only later did workers using a protein purification strategy identify the mammalian homolog of Swiss Cheese as the target of degeneration-inducing organophosphorus esters (Lush et al.(1998)). This is exciting because two completely different approaches lead to the same protein and because the Swiss cheese phenotype strongly suggests that the mammalian Swiss cheese gene, encoding the neuropathy target esterase, may lead to neural degeneration when mutated (Lush et al. (1998)).

An additional point that can be taken from Table 1 is that Drosophila mutants and phenotypes can be used to model and test various potential therapies. Specifically, the bubblegum mutation leads to accumulation of very long chain fatty acids, as is also seen in human adrenoleukodystrophy (ALD). The mutant phenotype can be rescued with glycerol trioleate oil, a component of “Lorenzo's oil” used to treat ALD.

Drosophila also offers an array of experimental tools which aid characterization of mutant phenotypes and gene function. Clones of mutant cells can be created in an otherwise wild type background and these mutant cells and their neural projections marked with easily scorable tags such as β-galactosidase or GFP (e.g. Lee, T. and Luo, L. (1999) Neuron. 22:451-461, incorporated by reference herein). This allows identification of abnormalities in the development or survival of specific mutant cells and detection of neural phenotypes that are cell autonomous or associated with small groups of closely related cells. The GAL4-UAS system (Brand, A. H. and Perrimon, N. (1993) Development. 11:401-415, incorporated by reference herein) and various enhancer traps, allow particular subsets of cells (e.g. glia, mushroom body neurons, photoreceptor neurons, etc.) to be marked in wild type or mutant backgrounds. This makes it possible to follow these labeled cells without having to sort through the other cells present in the brain. The GAL4-UAS system can also be used to express a wild type protein in a subset of cells in an otherwise mutant background and to mark these wild type cells so as to follow their development. This allows one to determine the extent that a mutant phenotype is caused by alterations in nearby cells as opposed to being caused by cell autonomous defects.

kelch was originally identified as a mutation altering egg morphology (Schupbach, T. and Wieschaus, E. (1991) Genetics 129:1119-1136, incorporated by reference herein). Further study showed that kelch function is necessary to maintain ring canals (incompletely closed contractile rings connecting the 15 nurse cells to each other and ultimately to the developing oocyte) during oocyte development. In the absence of Kelch, the ring canals form but components of the contractile ring, including actin, partially block the opening between cells, thereby blocking transport of key materials into the future egg (Robinson et al., (1994) Development 120:2015-25, incorporated by reference herein).

In the course of egg development, the precursor cell to the oocyte undergoes four rounds of division to create 16 cells. One of these becomes the oocyte, the other 15 are the nurse cells, which synthesize both proteins and RNAs for transport and storage in the oocyte. The divisions leading to the production of the oocyte are unusual in that cell division is not complete, the cells remain connected by bridges of cytoplasm. The material that the nurse cells will supply to the oocyte is transported through these cytoplasmic connections. The outside of these bridges, the ring canal, is a moderately complicated structure containing actin and a number of other proteins and has been used as a model for the construction of actin-based structures (see Robinson, D. N. and Cooley, L. (1997). Annu Rev Cell Dev Biol 13:147-70 for review, incorporated by reference herein).

Ring canals are assembled sequentially from arrested mitotic cleavage furrows. During cytokinesis, the contractile ring contains both anillin and contractile actin filaments. Later, phosphotyrosine, or at least material that reacts with anti-phosphotyrosine antibodies, appears at the outer rims of the ring canals, although the protein that reacts with the anti-phosphotyrosine has not been identified. This process is likely to depend on the protein kinases Tec29 and Src64 (Sokol and Cooley, 1999). After the final round of cell division, the inner and outer rims of the ring canal begin to form. First, in a process requiring the fillamin protein Cheerio, both actin and the protein Hts are recruited to the inner rim of the ring canal (Li, et al. (1999) J Cell Biol 146:1061-74; Sokol, N. S. and Cooley, L. (1999). Curr Biol 9:1221-30, each herein incorporated by reference). The hts gene encodes a set of proteins, some of which have homology to adducin. The ring canal specific form of Hts (Hts-RC) is derived from an RNA encoding a protein with N-terminal homology to vertebrate adducin and no homology in the C-terminus. The Hts-RC protein arises from the C-terminal portion of the protein encoded by the hts message (Robinson et al., 1994). After Hts-RC is incorporated into the inner rings, Kelch protein is added. In the absence of Hts-RC, Kelch is not added to the ring canals. Kelch is not required for assembly of the ring canal, but is required for later stability, growth, expansion and organization. The assembled ring canal contains the phosphotyrosine protein at its outer rim. The inner rim contains the phosphotyrosine protein, actin, Hts-RC and Kelch.

A critical point about ring canals is that they are dynamic and grow through development. If a ring canal is thought of as having a shape similar to a tire, the diameter can be defined as the distance from one outer edge of the canal to the other. Thickness is the distance from the inner rim to the outer edge. The length is the distance, parallel to the axis of the canal, from one edge of the ring to the other. Ring canals grow substantially in diameter and in length, even after stage 5, while the thickness remains relatively constant after this time. All of this occurs while maintaining a relatively constant density of actin filaments (Tilney, et al. (1996) J Cell Biol 133:61-74, incorporated by reference herein). This indicates that a substantial amount of actin must be added to the ring while maintaining the general aspects of the structure. In the absence of Kelch, the inner rings are disorganized, with changes in actin filament organization occurring as early as stage 6. Actin filament bundles and Hts-RC come away from the ring and enter the inner space (Robinson et al., 1994; Tilney et al., 1996). It has been suggested that Kelch functions by forming dynamic cross links to actin and potentially other proteins, allowing growth of the ring canals while maintaining a common basic structure (Robinson and Cooley, 1997; Tilney et al., 1996).

Drosophila kelch is only one member of the kelch repeat superfamily (Adams et al. Trends in Cell Biology, (2000) 10:17-24, incorporated by reference herein). Two human proteins closely related to Kelch have been identified, including one that is expressed predominantly in the brain in glia and neurons (Soltysik-Espanola et al., (1999). Although the structural characteristics of this family are currently being developed, not much is known about the biological and functional characteristics associated with this family of proteins.

### BRIEF DESCRIPTION OF THE INVENTION

In accordance with the present invention, it has been determined that Kelch, and its human homolog, Mayven, are critical to proper neural function, especially as animals age. Based on this discovery, methods of maintaining and/or restoring proper neural function, as well as compositions useful therefor, have been developed. In addition, there are provided transgenic animals useful for the study of age-related neurodegeneration.

### BRIEF DESCRIPTION OF THE DRAWINGS

FIG. 1 is a schematic diagram showing the structure of Kelch and positions of kelch mutations. The positions of the single BTB domain and six kelch repeats are indicated. Mutant alleles and the corresponding protein changes are indicated. Mutants 37, 75-004, 5 and 53 were isolated as described herein. DE1 and WB6 were supplied by Lynn Cooley. DE1 is a nonsense mutation and should be a null. Mutants 75-004, 5, WB6 and 53 all disrupt a conserved Hydrophobic-Gly-Gly motif conserved in kelch repeats.

FIGS. 2A and 2B are graphs illustrating age dependence of alterations in kelch behavior.

In FIG. 2A, the time to copulation was measured at the ages shown for wild type and kelch mutant females kept at 22° C.

In FIG. 2B, the time to copulation was measured at the ages shown for wild type and kelch mutant males kept at 22° C.

FIG. 3 is a graph illustrating the correlation of temperature and behavior. Temperature increases are seen to increase the speed of behavioral changes in kelch mutants. The time to copulation was determined for wild type and kelch females kept at 22° C. or 29° C. for one week as adults.

FIG. 4 is a graph showing the changes in wing position with aging in kelch mutants. Comparisons were between wild type and kelch mutants of the fraction of animals with dropped wings as a function of time at 29° C. as adults.

FIG. 5 presents data which indicates that kelch mutants have normal viability. Thus, wild type and kelch mutant animals were tested for viability as adults at 29° C.

### DETAILED DESCRIPTION OF THE INVENTION

The sum of the kelch phenotypes in oocyte development, plus the observation of changes in behavior in aging kelch mutant animals as disclosed herein, establish that Kelch acts in neurons as part of dynamic processes involving reorganization of actin-containing structures. The studies on ring canals also point to key additional genes that might also be involved in maintaining actin-based neural structures with aging. In addition, although the behavioral phenotypes associated with loss of kelch are not directly caused by abnormalities in the development of the oocyte, the findings herein on the role of kelch in female fertility elucidate how the Kelch protein family may function in the brain.

A genetic screen for alterations in behavior showed that mutations in the Drosophila kelch gene lead to age-dependent deterioration in sexual receptivity, mating success and body control without affecting life span. Studying sexual behavior and neural development elucidates aging research by studying this phenomena at multiple levels: behavioral and phenotypic characterization of molecularly mapped mutant alleles; characterization of neural defects at the whole brain level and at the level of defined neurons; determination of the relationship between Kelch expression patterns and the mutant phenotype; and preparation for more detailed cellular, biochemical and neurological characterization of the role of Kelch in normal functions. The experimental amenability of the system and existence of close human homologs make this gene potentially directly relevant to aging, particularly human aging.

This disclosure characterizes key aspects of aging dependent neural and behavioral changes associated with mutations in the kelch gene of Drosophila. Kelch is an actin binding and organizing protein originally identified based on phenotypes resulting from abnormal transfer from nurse cells to the developing oocyte. This work shows that kelch also acts in somatic tissues. In the absence of kelch function, both males and females show substantial, unusual changes in behavior as they age. Females stop laying eggs, and become resistant to males before and during copulation. Males lose the ability to bend their abdomen to the extent necessary for efficient copulation. Both sexes lose the ability to maintain the wings in the normal resting position on top of the back and no longer move them efficiently. Interestingly, unlike many Drosophila neurodegeneration mutations, these phenotypes are not obviously associated with premature death. Kelch mutants live as long as wild type but lose the ability to do many things associated with normal activities.

In one embodiment, of the present invention, methods for using substantially pure kelch polypeptide and functional fragments thereof are provided. As disclosed herein, the term “Kelch” or “kelch” refers to members of the kelch repeat superfamily of proteins, including the mammalian homologue also known as mayven (see, e.g., Adams et al. (2000). Invention polypeptides are useful as immunogens for producing antibodies which bind to a Kelch polypeptide or functional fragment thereof.

In yet another embodiment of the present invention, transgenic animals (including insects) having a transgene disrupting expression of Kelch, chromosomally integrated into the cells of the insect are provided. Nucleic acid constructs including a disrupted kelch gene, such that the disruption prevents expression of functional kelch polypeptide, and host cells transformed therewith, also are provided.

In still another embodiment of the present invention, transgenic animals having a transgene encoding a member of the Kelch family or functional fragment thereof are provided. Methods are also provided for producing invention transgenic animals, said methods comprising introducing into the genome of an insect invention polynucleotides encoding Kelch polypeptide (or functional fragment thereof) operatively linked to a promoter which functions in insect cells to cause the production of an RNA sequence, and obtaining a transgenic insect having a nucleic acid encoding Kelch (or functional fragment thereof). There are also provided transgenic animals and methods of production thereof, which have an allogeneic or xenogeneic source for the transgene.

In another series of embodiments of the present invention, there are provided transgenic animal models for neurodegenerative diseases, including age dependent neural degenerative diseases associated with mutations in the kelch genes. The animal may be essentially any mammal, including rats, mice, hamsters, guinea pigs, rabbits, dogs, cats, goats, sheep, pigs, and non-human primates. In addition, invertebrate models, including nematodes and insects, may be used for certain applications (Min & Benzer (1999) Science 284:1985-2375, incorporated by reference herein). The animal models are produced by standard transgenic methods including microinjection, transfection, or by other forms of transformation of embryonic stem cells, zygotes, gametes, and germ line cells with vectors including genomic or cDNA fragments, minigenes, homologous recombination vectors, viral insertion vectors, and the like. Suitable vectors include vaccinia virus, adenovirus, adeno associated virus, retrovirus, liposome transport, neuraltropic viruses, Herpes simplex virus, and the like. The animal models may include transgenic sequences comprising or derived from Kelch, including normal and mutant sequences, intronic, exonic and untranslated sequences, and sequences encoding subsets of Kelch such as functional domains.

The major types of animal models provided include: (1) Animals in which a normal human kelch (mayven) gene has been recombinantly introduced into the genome of the animal as an additional gene, under the regulation of either an exogenous or an endogenous promoter element, and as either a minigene or a large genomic fragment; in which a normal human kelch gene has been recombinantly substituted for one or both copies of the animal's homologous kelch gene by homologous recombination or gene targeting; and/or in which one or both copies of one of the animal's homologous kelch genes have been recombinantly “humanized” by the partial substitution of sequences encoding the human homologue by homologous recombination or gene targeting. (2) Animals in which a mutant human ketch gene has been recombinantly introduced into the genome of the animal as an additional gene, under the regulation of either an exogenous or an endogenous promoter element, and as either a minigene or a large genomic fragment; in which a mutant human kelch gene has been recombinantly substituted for one or both copies of the animal's homologous kelch gene by homologous recombination or gene targeting; and/or in which one or both copies of one of the animal's homologous kelch genes have been recombinantly “humanized” by the partial substitution of sequences encoding a mutant human homologue by homologous recombination or gene targeting. (3) Animals in which a mutant version of one of that animal's kelch genes has been recombinantly introduced into the genome of the animal as an additional gene, under the regulation of either an exogenous or an endogenous promoter element, and as either a minigene or a large genomic fragment; and/or in which a mutant version of one of that animal's kelch genes has been recombinantly substituted for one or both copies of the animal's homologous kelch gene by homologous recombination or gene targeting. (4) “Knock-out” animals in which one or both copies of one of the animal's kelch genes have been partially or completely deleted by homologous recombination or gene targeting, or have been inactivated by the insertion or substitution by homologous recombination or gene targeting of exogenous sequences.

In presently preferred embodiments of the present invention, a transgenic animal model for age associated neurodegenerative disease has a transgene encoding a normal mammalian mayven protein, a mutant mammalian kelch protein, or a humanized normal or mutant kelch protein generated by homologous recombination or gene targeting.

In accordance with yet another aspect of the present invention, there are also provided methods for identifying compounds that modulate Kelch activity or expression of a polynucleotide encoding Kelch, said methods comprising incubating components comprising a test compound, and a Kelch polypeptide (or functional fragment thereof), or a cell expressing a Kelch polypeptide (or functional fragment thereof), under conditions sufficient to allow the components to interact, and detecting an effect of the test compound on Kelch polypeptide activity or expression of a polynucleotide encoding kelch. Such compounds, including agonists and antagonists of Kelch activity or expression of a polynucleotide encoding Kelch, can be useful for modulating Kelch biological activities. Candidate test compounds include insect hormones, libraries thereof, and combinatorial libraries.

In another series of embodiments of the present invention, there are provided methods of screening or identifying proteins, small molecules or other compounds which are capable of inducing or inhibiting the expression of the kelch genes and proteins. The assays may be performed in vitro using transformed or non-transformed cells, immortalized cell lines, or in vivo using the transgenic animal models or human subjects enabled herein. In particular, the assays may detect the presence of increased or decreased expression of Kelch or other kelch-related genes or proteins on the basis of increased or decreased mRNA expression, increased or decreased levels of kelch-related protein products, fragments thereof such as a kelch repeat, or increased or decreased levels of expression of a marker gene (e.g., beta-galactosidase, green fluorescent protein, alkaline phosphatase or luciferase) operably joined to a kelch 5′ regulatory region in a recombinant construct. Cells known to express a particular Kelch, or transformed to express a particular kelch, are incubated and one or more test compounds are added to the medium. After allowing a sufficient period of time (e.g., 0-72 hours) for the compound to induce or inhibit the expression of the kelch, any change in levels of expression from an established baseline may be detected using any of the techniques described above. In particularly preferred embodiments, the cells are from an immortalized cell line such as a human neuroblastoma, glioblastoma or a hybridoma cell line, or are transformed cells of the invention.

In another series of embodiments of the present invention, there are provided methods for identifying proteins and other compounds which bind to, or otherwise directly interact with, Kelch. The proteins and compounds contemplated for identification herein will include endogenous cellular components which interact with kelch in vivo and which, therefore, provide new targets for pharmaceutical and therapeutic interventions, as well as recombinant, synthetic and otherwise exogenous compounds which may have presenilin binding capacity and, therefore, may be candidates for pharmaceutical agents.

Thus, in one series of embodiments, cell lysates or tissue homogenates (e.g., human brain homogenates, lymphocyte lysates) may be screened for proteins or other compounds which bind to normal and/or mutant Kelch. Alternatively, any of a variety of exogenous compounds, both naturally occurring and/or synthetic (e.g., libraries of small molecules or peptides), may be screened for kelch binding capacity. In each of these embodiments, an assay is conducted to detect binding between a “Kelch component” and some other moiety. The “Kelch component” in these assays may be any polypeptide or polynucleotide comprising or derived from a normal or mutant Kelch protein/nucleotide, including functional domains or antigenic determinants of Kelch, or Kelch fusion proteins. Binding may be detected by non-specific measures (e.g.,changes in biological or phenotypic activity, changes in the expression of other downstream genes which can be monitored by differential display, 2D gel electrophoresis, differential hybridization, or SAGE methods) or by direct measures such as immunoprecipitation, the Biomolecular Interaction Assay (BlAcore) or alteration of protein gel electrophoresis. The presently preferred methods involve variations on the following techniques: (1) direct extraction by affinity chromatography; (2) co-isolation of presenilin components and bound proteins or other compounds by immunoprecipitation; (3) BlAcore analysis; and (4) yeast two-hybrid systems.

In another series of embodiments of the present invention, there are provided methods of identifying proteins, small molecules and other compounds capable of modulating the activity of normal or mutant Kelch. Using normal cells or animals, the transformed cells and animal models of the present invention, or cells obtained from subjects bearing normal or mutant kelch genes, the present invention provides methods of identifying such compounds on the basis of their ability to affect the expression of Kelch, the intracellular localization of Kelch, or other ion levels or metabolic measures, or other biochemical, histological, or physiological markers which distinguish cells bearing normal and mutant kelch sequences. Using the animal models of the invention, methods of identifying such compounds are also provided on the basis of the ability of the compounds to affect behavioral, physiological or histological phenotypes associated with mutations in Kelch.

Further provided are agricultural compositions comprising invention polypeptides, polynucleotides, antibodies, transgenic insects and compounds that modulate Kelch activity or expression of a polynucleotide encoding Kelch. Insecticidal compositions include transgenic insects having disrupted expression of Kelch and compounds that modulate Kelch activity or expression of a polynucleotide encoding Kelch which are useful for effecting control of insect pests. In one embodiment, an insecticidal composition contains a transgenic insect carrying a transgene comprising DNA disrupting expression of Kelch and an agriculturally acceptable carrier is provided. In another embodiment, a transgenic insect carrying a transgene comprising DNA disrupting expression of a nucleic acid encoding Kelch, in a manner such that these polynucleotides are stably integrated into the DNA of germ line cells of the mature insect and inherited in normal Mendelian fashion, is released into the environment. The insect can then mate with insects in the environment, such that the progeny will carry DNA disrupting expression of nucleic acid sequence encoding Kelch. After two or more generations of the transgenic insects mating with the insects in the environment, altered Kelch homozygous progeny insects will be produced which exhibit characteristics associated with altered Kelch as described herein.

In another embodiment of the present invention, there are provided insecticidal compositions containing an invention polynucleotide or polypeptide that inhibits or prevents a Kelch biological activity or function. Such compositions can be used as a component of an agricultural composition for applying to plants, plant environments, or distributed in baits to effect insecticidal control of an insect. For example, a DNA disrupting expression of Kelch polynucleotide, a double-stranded RNAi molecule, an antisense Kelch polynucleotide or a polynucleotide encoding dominant negative Kelch, in a vector if appropriate, can be contained in an insecticidal composition. The target insect guides one of skill in the art in the selection of the agent for insect control.

In still yet another embodiment of the present invention, there are provided insecticidal compositions which contain an invention compound which modulates Kelch biological activity or expression of a Kelch polypeptide. For example, Kelch antagonists that inhibit Kelch biological activity or expression of a Kelch polypeptide can confer a Kelch phenotype on an insect thereby reducing or preventing reproduction. Such antagonists can function to decrease Kelch biological activity, Kelch synthesis (transcription or translation) or Kelch stability (transcript or polypeptide).

Also provided in accordance with the present invention are agricultural compositions that promote or activate a Kelch biological activity or function, including, for example, invention compounds that modulate a Kelch biological activity or activate a Kelch biological activity or expression of a Kelch polypeptide in insect cells not normally expressing active Kelch (i.e., misexpression). Such compounds can be used to effect insecticidal control of an insect. Such agonists can function to increase Kelch biological activity, Kelch synthesis (transcription or translation) or Kelch stability (transcript or polypeptide). In another embodiment, such agricultural compositions contain a nucleic acid encoding Kelch and an agriculturally acceptable carrier.

In another embodiment of the present invention, there are provided agricultural compositions comprising a transgenic insect carrying a transgene comprising a nucleic acid encoding Kelch (or functional fragment thereof) operatively linked to an expression control element and an agriculturally acceptable carrier. In one aspect, a conditional promoter drives Kelch expression. In another aspect, an expression control element controlling expression in a manner substantially similar to Kelch polypeptide expression drives expression. As transgenic insects so transformed may have increased reproductive capability as a result of increased egg laying by females, for example, beneficial insects (e.g., those that pollinate plants or produce useful products, foodstuffs, and the like, such as honeybees) and predatory insects (e.g., ladybugs, praying-mantis, walking sticks, assassin bugs, and the like) expressing a Kelch transgene (or functional fragment thereof) may exhibit increased proliferation. Such beneficial insects can provide increased foodstuff production or for the insecticidal control of insect pests, as appropriate. Preferably, such transgenic insects do not contain altered Kelch or other genes required for normal reproductive function.

The concentration of the aforementioned agricultural compositions required to be effective will depend on the type of organism targeted and the formulation of the composition and the effect on reproductive behavior or function desired (i.e., increased or decreased). For example, an insecticidally effective agricultural composition is that amount sufficient to cause a significant reduction in an insect population. The phrase “insecticidally effective” means an amount sufficient to cause a significant reduction in an insect population. The insecticidally effective concentration can be readily determined experimentally by one of skill in the art.

Invention agricultural compositions must be suitable for agricultural use and dispersal in fields. Similarly, compositions for the control of insect pests must be environmentally acceptable. Generally, components of the composition must be nonphytotoxic and not detrimental to the integrity of the virus vector. Foliar applications must not damage or injure plant leaves. In addition to appropriate solid or, more preferably, liquid carriers, agricultural compositions may include sticking and adhesive agents, emulsifying and wetting agents, but not components which deter insect feeding or viral functions. It may also be desirable to add components which protect the insecticidal composition from UV inactivation, degradation or components which serve as adjuvants. Reviews describing methods of application of biological insect control agents and methods and compositions for agricultural application are available (see e.g., Couch and Ignoffo, In: Microbial Control of Pests and Plant Disease 1970-1980, Burges (ed.), chapter 34, pp. 621-634, 1981; Corke and Rishbet, ibid, chapter 39, pp. 717-732; Brockwell, In: Methods for Evaluating Nitrogen Fixation, Bergersen (ed.), pp. 417-488, 1980; Burton, In: Biological Nitrogen Fixation Technology for Topical Agriculture, Graham and Harris (eds.), pp. 105-114, 1982; Roghley, ibid, pp. 115, 127, 1982; and The Biology of Baculoviruses, Vol. 11, Biological Properties and Molecular Biology, CRC Press, Inc. Boca Raton, Florida, 1986, each of which are incorporated by reference herein).

In another series of embodiments of the present invention, there are provided methods for screening for carriers of Kelch alleles associated with age-associated neurodegenerative diseases. Such methods can be employed for a variety of purposes, such as, for example, for diagnosis of victims of such disorders, for the screening and diagnosis of related diseases, including dementias, psychiatric diseases such as schizophrenia and depression, decreased sexual ability and desire, decreased motor skills and other neurologic diseases, disorders and behaviors associated with age, and the like. Screening and/or diagnosis can be accomplished by methods based upon the nucleic acids (including genomic and mRNA/cDNA sequences), proteins, and/or antibodies disclosed and enabled herein, or known to those skilled in the art, including functional assays designed to detect failure or augmentation of the normal Kelch activity and/or the presence of specific new activities conferred by mutant Kelch. Thus, screens and diagnostics based upon presenilin proteins are provided which detect differences between mutant and normal presenilins in electrophoretic mobility, in proteolytic cleavage patterns, in molar ratios of the various amino acid residues, and in ability to bind specific antibodies. In addition, screens and diagnostics based upon nucleic acids (gDNA, cDNA or mRNA) are provided which detect differences in nucleotide sequences by direct nucleotide sequencing, hybridization using allele specific oligonucleotides, restriction enzyme digest and mapping (e.g., RFLP, REF-SSCP), electrophoretic mobility (e.g., SSCP, DGGE), PCR mapping, RNase protection, chemical mismatch cleavage, ligase-mediated detection, and various other methods. Other methods are also provided which detect abnormal processing of Kelch, or proteins reacting with Kelch (e.g., abnormal phosphorylation, glycosylation, glycation amidation or proteolytic cleavage), alterations in Kelch transcription, translation, and post-translational modification; alterations in the intracellular and extracellular trafficking of presenilin gene products; or abnormal intracellular localization of the presenilins. In accordance with these embodiments, diagnostic kits are also provided which will include the reagents necessary for the above-described diagnostic screens.

In another series of embodiments of the present invention, there are provided methods and pharmaceutical preparations for use in the treatment of Kelch-associated diseases such as age-dependent neural degenerative diseases. These methods and pharmaceuticals are based upon (1) administration of normal Kelch proteins, (2) gene therapy with normal Kelch genes to compensate for or replace the mutant genes, (3) gene therapy based upon antisense sequences to mutant Kelch genes or which “knock-out” the mutant genes, (4) gene therapy based upon sequences which encode a protein which blocks or corrects the deleterious effects of Kelch mutants, (5) immunotherapy based upon antibodies to normal and/or mutant Kelch proteins, or (6) small molecules (drugs) which alter Kelch expression, block or enhance abnormal interactions between mutant forms of Kelch and other proteins or ligands, or which otherwise block or enhance the aberrant function of mutant Kelch proteins by altering the structure of the mutant proteins, by enhancing their metabolic clearance, or by inhibiting their function, or by enhancing the decreased interaction of mutant forms of Kelch and other proteins or ligands, and the like.

The following examples are intended to illustrate but not limit the invention in any manner, shape, or form, either explicitly or implicitly. While they are typical of those that might be used, other procedures, methodologies, or techniques known to those skilled in the art may alternatively be used.

### EXAMPLE 1

### Genetic and Molecular Localization of Dissatisfaction

In accordance with the present invention, it has been discovered that mutations in the kelch gene generate aging-dependent alterations of male and female sexual behavior and performance, with young animals being essentially normal and older animals showing striking changes in behavior and decreases in sexual performance. Based on the initial wild type behaviors and their later deterioration, it can be concluded that kelch is unlikely to be a key regulator of the development of the sex-specific nervous system, but is very probably required for normal function during aging of those neurons needed for sexual behaviors, and perhaps others. This leads one directly into studies of the age associated kelch neural phenotypes and the mechanism of function of kelch in maintaining normal neural function. The techniques and assays described herein are similar to those developed or applied in the studies of genetic control of sexual behavior (Finley, et al. (1997) Proc. Natl. Acad. Sci. USA. 94:913-913; Finley, et al. (1998) Neuron. 21:1363-1374, each herein incorporated by reference). These techniques are now being applied to a set of biomedical questions and problems that represent a major shift in research focus, evolving from one focus (sexual behavior) to a notably different one (changes in behavior with aging).

Initial Identification of Behavioral Abnormalities Associate with Kelch Mutations

As part of continuing studies of genes controlling sexual differentiation and sex-specific behaviors, a genetic screen was developed for mutations altering female sexual behavior. This screen was initially suggested by the phenotype of an unusual allele of transformer, an upstream gene in the sex differentiation cascade, and has been validated by the identification of the dissatisfaction (dsf) gene (Finley et al. (1997), (1998)), and one additional locus (unpublished), both of which disrupt female and male sexual behavior and sex-specific neurons while leaving other functions intact. Briefly, the screen begins with identification of female-sterile mutations, including those that disrupt egg laying, a key female-specific behavior. This small pool of candidate loci is then tested for abnormalities in female and male sexual behaviors including changes in receptivity (e.g. do females become receptive to males with normal kinetics, as judged by time of courtship prior to copulation; do they stand quietly during copulation or do they try to dislodge the male?); changes in sex partner choice (e.g. do males court males as well as females?); and changes in copulation efficiency (e.g. do males mate efficiently with receptive females as judged by time to copulation; can they efficiently perform the 180° abdominal bend necessary to initiate copulation?).

In the course of such a screen, four alleles of a single locus were identified, initially denoted 75-004. Using homozygous animals taken from stock, eggs of normal or near normal size were often observed stuck in the uterus (similar to dsf). In mating assays, some mutant females were extremely aggressive in resisting males during copulation, including even bucking off copulating males, an extremely unusual phenotype. Some mutant males showed striking deficiencies in copulation, including notable difficulties in bending the abdomen the full 180° necessary to copulate.

Recombination mapping followed by deletion mapping localized 75-004 to a genetically small region containing the kelch locus. Complementation testing for female fertility between 75-004 and kelch revealed that 75-004 is an allele of kelch. Since it was a formal possibility that the kelch mutation on the 75-004 chromosome was not the mutation causing the behavioral deficits, 75-004/kelch heterozygotes were tested for mating behavior and at least some males and females were observed showing phenotypes similar to 75-004 homozygotes.

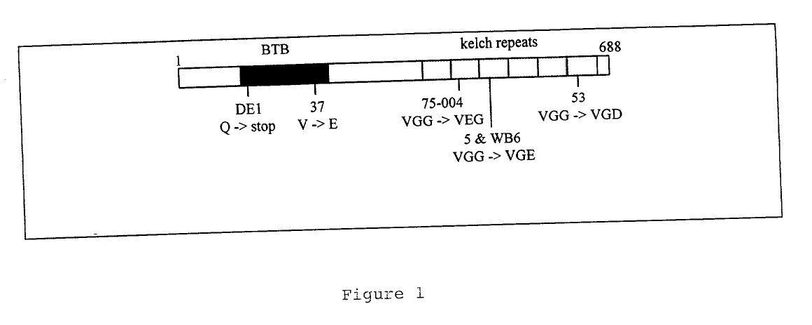

As noted above, kelch was originally identified as a mutation altering egg morphology. As noted in FIG. 1, Kelch contains an N-terminal BTB domain (a protein-protein interaction domain) and a C-terminal set of six “Kelch repeats” which are probably involved in association with actin (Robinson, D. N. and Cooley, L. (1997) J Cell Biol. 138:799-810, each herein incorporated by reference).

To verify that 75-004 and other alleles isolated during the above-described screen were indeed mutations in kelch, and to determine the nature of the lesions, the protein-coding DNA of all 4 new alleles was PCR amplified and sequenced (FIG. 1). Each of the new alleles contains a single nucleotide change from the parental DNA and each nucleotide change leads to a key amino acid change. One of the new alleles alters an amino acid in the BTB domain, the others alter three different kelch repeats, in each case changing one of a pair of Gly residues conserved in most kelch repeats. Two kelch alleles (generously supplied by Lynn Cooley (Yale)) were also sequenced. One of these two alleles changes the same kelch repeat Gly residue as one of the new alleles, while the other is a nonsense mutation early in the coding sequence and, therefore, likely to be a true null.

The involvement of Kelch in ovary function raises the obvious question of “what does egg development have to do with neural degeneration or sexual behavior?” The answer is probably “nothing at all.” Rather it is likely that Kelch, like many other proteins, is involved in multiple functions. One function is maintenance of ring canal structure while another is maintenance of some important aspect of neuronal structure or function. Both functions are likely to involve interactions with the actin cytoskeleton. In support of this hypothesis, western blotting shows that Kelch is present in multiple tissues, including a pool of brain tissue and imaginal discs (the precursors of the adult cuticle) (Robinson, D. N. and Cooley, L. (1997) Development 124:1405-17, incorporated by reference herein).

Thus, at the conclusion of the earliest of the studies described herein, kelch had been identified as a gene with a potential function in generating or maintaining normal sex-specific neural functions. In addition, a collection of molecularly mapped kelch mutations had been developed, including a null mutant and four different domain-specific mutations.

Phenotypic Characterization of Kelch Mutants

Relatively early in the present studies variabilities in phenotypic penetrance or severity of the above-described kelch mutants were noted. Sometimes females would lay lots of eggs with kelch morphology, but on other occasions they laid no eggs and accumulated slightly small to nearly full sized eggs in the ovaries and uterus. Sometimes females were extremely and aggressively resistant to males, especially during copulation, other times they showed no differences from wild type. Sometimes males copulated with ease, other times they showed serious defects in the abdominal bending necessary for copulation. Examination of the data and the conditions of the experiments suggested that the age of the animals being tested substantially altered the severity of the phenotype.

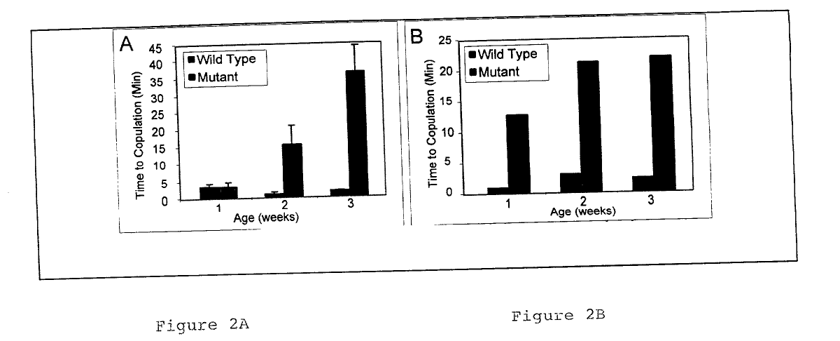

As a direct test of this hypothesis, a series of experiments was initiated to characterize kelch phenotypes as flies age. FIG. 2A shows data on the time spent courting prior to copulation for wild type and kelch females, a measure of female receptivity or resistance to males (Finley (1997), (1998)). As can be seen, wild type females copulate quickly at all ages while kelch females show increasing resistance. Mating efficiency among kelch males was also examined (FIG. 2B). Mutant males are inefficient in courtship and mating, and the trend is toward greater deficits at advanced ages. Some of this is the result of decreased efficiency in abdominal bending, making copulation difficult. This is also reflected in a tendency of the male's torso to pop away from the female's body during copulation. As males age more, not only is abdominal bending inefficient, but the ability to control wing position appears to decline (see FIG. 3) and the males no longer generate a courtship song.

In order to facilitate the analysis, the possibility of increasing the rate of phenotypic deterioration in kelch mutants was tested by raising the temperature to 29° C. once the animals eclose as adults. Others have shown that this accelerates aging (Min, K. T. and Benzer, S. (1999) Science. 284:1985-1988, incorporated by reference herein). FIG. 3 shows that such a temperature shift speeds the development of female resistance.

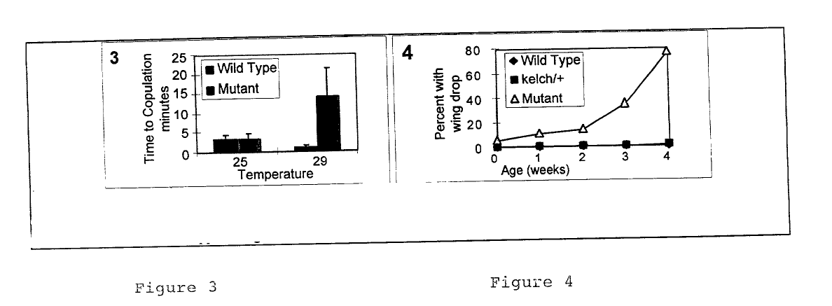

Potential neural or neuromuscular phenotypes not associated with sexual behavior are also observed. For example, kelch mutants eclose with normal morphology and body carriage. As they age their wings drop down and away from the body until nearly 100% of the animals show the phenotype. FIG. 4 shows a plot of such data for animals kept at 29° C. This is also seen at lower temperatures.

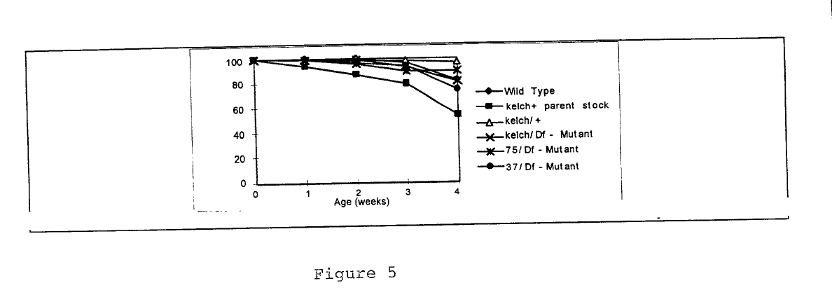

The kelch mutants described herein are different from many of the Drosophila neurodegeneration mutants in that they were selected for alterations in behavior and not for decreased viability (Min & Benzer (1997), (1999); Rogina, et al. (1997) Proc Natl Acad Sci USA. 94:6303-6306, each herein incorporated by reference). This suggested the desirability of testing to see if kelch leads to notable decreases in life span or if the behavioral deficits occur in the absence of gross changes in viability. FIG. 5 shows that kelch mutants show similar viability to wild type controls at 29° C., unlike mutants such as spongecake and eggroll that show 50% death after 10 days at 29° C. (Min & Benzer (1997) Curr Biol 7:885-888, incorporated by reference herein). Thus, in spite of substantial functional deficits, kelch animals have a near normal life span.

Although kelch leads to abnormalities in sexual behavior, the phenotypes are notably different from those seen with sex behavior genes such as dsf (Finley (1997), (1998)). For example, dsf mutant females are resistant to males from an early age, dsf mutant males copulate poorly from an early age, and the dsf phenotypes do not appear to extend to non-sexual behaviors. The age dependence of the kelch phenotypes, and the extension of kelch phenotypes to other areas, such as body carriage, strongly suggest that kelch is not directly involved in the control of sex-specific nervous system development. Rather, the phenotypes suggest that kelch normally functions to maintain the integrity of neurons or neural circuits involved in a number of different functions, the most easily assayed of which is sexual behavior. This is particularly exciting possibility because kelch substantially lowers the ability to perform functions associated with a normal, active life without leading to grossly premature death, just as some human diseases have similar effects on quality, rather than length of life.

Potential Human Homologs

Given the interesting nature of the kelch phenotypes, regular scanning of the sequences in GenBank for new proteins related to Kelch reveals two closely related proteins to Kelch; both are derived from humans. Mayven (Soltysik-Espanola, et al. (1999) Mol Biol Cell. 10:2361-2375, incorporated by reference herein) shows high level homology (63% identity, 77% similarity) from the BTB domain through to the extreme C-terminus of the protein. AC004021 (an unpublished sequence derived from a human PAC) begins near the end of the BTB domain and then matches well (60% identity, 77% similarity) to the C-terminus of the protein. Mayven is predominantly expressed in the brain, localizes with actin in astrocytoma/glioblastoma cell lines, is in the cell bodies and processes of cultured hippocampal neurons, and redistributes upon depolarization (Soltysik-Espanola, et al. (1999)). As with Kelch, the BTB domains of Mayven appear to be involved in self-oligomerization and actin binding (Robinson & Cooley (1997); Soltysik-Espanola, et al. (1999)). It is interesting that the expression pattern and biochemical functions of Mayven are completely consistent with the proposed role of Kelch in preventing neurodegeneration.

kelch mutants were isolated on the basis of adult behavioral alterations in the absence of grossly premature death. All of the behavioral deficits studied increase with age suggesting that Kelch normally serves to maintain the integrity or function of neurons or neural circuits as animals age. A large array of molecularly defined mutant alleles exists allowing initial structure function tests. The recent identification of a human protein with structural and biochemical properties similar to Kelch that is expressed in neurons and glia assures that study of kelch mutants is directly relevant to human disease.

### EXAMPLE 2

### Behavioral Analysis of Molecularly Mapped kelch Alleles

The analysis of kelch mutant phenotypes described herein makes it clear that kelch has consequences for behavior and that the mutant phenotypes become more severe with age. On the other hand, there has been no testing for variation between mutant alleles across the array of observed phenotypes. Determining the phenotypic severity for kelch alleles is critical in determining possible functional domains of the protein, possible involvement of individual domains in different functions, possible consequences of similar changes in human Kelch homologs, and in identifying those alleles and tests that will be most useful in screening for second site mutations that exacerbate or suppress the kelch phenotype. In addition, work on other systems in which protein-protein interactions are involved has demonstrated that in some cases protein null alleles generate less complete phenotypes, as a result of erroneous cross talk from other pathways, than disabling mutations in which protein is still present (e.g. (Madhani, et al. (1997) Cell. 91:673-684, incorporated by reference herein). The molecular mapping of kelch alleles at five different sites in the protein (a STOP early in the sequence, and missense mutations in the BTB domain and in the second, third and sixth Kelch domains, FIG. 1) makes it likely that the phenotypic comparisons can be maximally informative. All four missense alleles are derived from the same starting chromosome as part of the same genetic screen. The null and missense chromosomes have the same genetic markers and are, within the kelch coding sequence, identical at the nucleotide level except for the mutation-associated base changes. Thus variation due to genetic background is minimized in the assays described herein.

Combinations of the parental chromosome (control) or the molecularly defined point mutants (experimental) and the small kelch mutant deletion Df(2L)H20 are employed. This minimizes genetic background differences, generates otherwise healthy, wild type animals, and focuses attention on phenotypes generated by the kelch mutants. To speed the analysis, all adults are kept at 29° C. from eclosion to testing.

Life span and wing drop are tested together. Males and females are collected within 24 hours of eclosion and placed, 10 per vial, in multiple separate vials. At two day intervals animals are transferred without anesthesia to vials with fresh food and the viable flies counted and scored for the drop wing phenotype.

Egg Laying

kelch was originally identified because mutant nurse cells fail to transfer their contents to the oocyte, leading to abnormalities in the morphology of eggs. In the course of the present study, it has been observed that relatively young mutant females lay mutant eggs while older females lay few or no eggs, instead accumulating eggs in the ovaries and in the uterus. To quantitate this phenomenon, and establish a time course, two sets of experiments are performed. 1) Virgin females are collected and aged for varying lengths of time, 2 days, 5 days, 7 days, 10 days, 14 days, etc. prior to adding wild type males. Since females normally lay few eggs prior to mating, and lay a relatively large number after mating, adding males at set times after collection of females allows one to define the time at which to expect maximum egg-laying activity. Starting with the addition of males, groups of females are transferred to fresh food every day and the number of eggs laid in each 24 hour period scored. 2) Since kelch mutant females become resistant to males before and during copulation, it is possible that any deficit in egg laying with age seen in (1) above results from failure to initiate or complete mating. For those time points and alleles showing a significant decrement in egg laying with age, or in conjunction with the next set of experiments below, individual pair matings are set up between aged mutant females and wild type males, and those pairs noted in which copulation occurs and extends for the wild type 17-20 minute period. Females that have copulated are then transferred to vials and scored for egg laying.

Female Receptivity to Courtship and Copulation

Virgin females are collected and aged as described above. Females of various ages are individually transferred to a “mating chamber” of about 20 mm diameter and 5 mm depth with a single wild type male. The time of courtship prior to copulation are scored as are the behavior of the female during copulation including movement (a correlate of resistance) bucking, wing flicking and kicking, as well as the total time of copulation (Finley (1997), (1998)). Mated females are then removed and tested for egg laying as in part (2) just above.

Male Courtship and Copulation Efficiency

Males are collected, aged appropriately and placed in mating chambers with 5-7 day old wild type virgin females. Time to copulation, an indicator of courtship and copulation efficiency (Finley (1997), (1998)) are scored. Visual and video analysis are used to determine if the males show efficient abdominal bending during attempted copulation (Finley (1997), (1998)).

### EXAMPLE 3

### The Nature of the Changes in the Nervous System in kelch Mutants

The initial results generated herein show, and the experiments above further quantitate, the key functional aspects of the kelch phenotype: Abnormal loss of function and behavior with age. If the above-described analysis is to be extended to the cellular and molecular levels, the underlying changes in the nervous system (and perhaps the muscular system) are to be defined and correlated with changes in the behavioral output of kelch mutants. The methods contemplated herein involve examination of whole brain morphology; examination of specific motor neuron projections, especially to muscles in the uterus and body wall involved in egg laying or mating; examination of specifically defined sets of CNS and sensory neurons; examination of marked glia; and examination of small groups of kelch mutant cells in a wild type background.

Analysis of Whole Brain Morphology

For a number of neurodegeneration mutants, abnormalities can be observed in the gross morphology of brain and nervous system tissue (e.g. (Buchanan, R. L. and Benzer, S. (1993) Neuron. 10:839-850; Kretzschmar et al. (1997), Min & Benzer (1997), (1999), each herein incorporated by reference). To determine if kelch leads to extensive brain degeneration or cell death, kelch mutant brains are examined at various ages for abnormalities and apoptosis. To check for bulk degeneration, adult heads are fixed, dehydrated, and embedded in paraffin embedding medium. Sections are cut, mounted on coated slides, and stained with Mayer hematoxylin or toluidine blue to reveal brain morphology (Restifo, L. L. and White, K. (1991) Dev Biol. 148:174-94, incorporated by reference herein). Neural degeneration, if present, is seen in the deterioration or shrinkage of normal brain structures, or development of vacuoles or inclusion bodies. To test for apoptosis, frozen head sections are prepared for immunohistochemistry and stained for apoptosis using an ApopTag kit from Oncor (Kretzschmar et al. (1997)). This leads to digoxygenin labeling of fragmented cellular DNA in cells undergoing apoptosis. These cells are then visualized using anti-digoxygenin antibodies and DAB staining.

Analysis of Motor Neuron Projections

Some kelch phenotypes such as loss of egg laying and deficits in abdominal bending are similar to those observed for other mutations such as dsf that lead to abnormalities in neuromuscular junctions on the uterus and ventral abdominal muscles (Finley (1997), (1998)). Anti-synaptotagmin antibodies are used to determine if the synapses on the uterus and ventral abdominal muscles are normal or abnormal and if the gross structure of the neuromuscular junctions changes with age. Similarly, with regard to the drop wing phenotype, the structure of synapses are examined on the muscles of the thorax which are largely responsible for wing positioning and movement.

Analysis of Specific Neurons and Glia in the CNS and Visual System

Although small, the Drosophila CNS is a complicated structure with up to 100,000 neurons. As a step toward analysis of individual neurons, or defined subsets of neurons, cell bodies are labeled and projections of defined subsets of cells are generated. Specifically, a system is used in which defined enhancers are used to express the yeast GAL4 protein in a specific pattern, and the GAL4 protein is then used to induce expression of an easily followed marker protein such as GFP or Tau-LacZ (1). A set of enhancer GAL4 stocks is currently available from Greenspan and from Kaiser (Ferveur, et al. (1995) Science. 267:902-905; O'Dell, et al. (1995) Neuron. 15:55-61; Yang, et al. (1995) Neuron. 15:45-54, each herein incorporated by reference) that target defined subsets of the CNS neurons, including but not exclusively, subsets of neurons in the antennal lobe and mushroom body (a structure involved in aspects of memory). In addition, an enhancer construct is also available that allows one to label differentiating cells of the eye, including the photoreceptor neurons and their projections into the first layers of the visual brain, the lamina (for the outer photoreceptors) and the medulla (for direct projection of the inner photoreceptors) (Hay, et al. (1994) Development. 120:2121-2129, incorporated by reference herein). When coupled to various GFP constructs, these lead to labeling of the cell bodies and full projection patterns for these neurons. Wild type and kelch mutant animals containing the components of this cell marking system are constructed and their brains examined at various times for abnormalities in structure, projections, or other properties.

Since primary defects leading to neurodegeneration can occur in the glia as well as in the neurons (e.g. (Buchanan & Benzer (1993), Halter, et al. (1995) Development. 121:317-32; Xiong, W. C. and Montell, C. (1995) Neuron. 14:581-90, each herein incorporated by reference), GAL4-UAS and enhancer trap systems are used to mark glial cells. Two different enhancer constructs are employed for this purpose. The first is a LacZ enhancer trap inserted in repo, which marks most glia (Xiong, et al. (1994) Genes Dev. 8:981-94; Halter et al. (1995); Min & Benzer (1997), each herein incorporated by reference). The second is the Alk enhancer fused to GAL4 which marks at least a subset of glia (unpublished work). As above and as in (15) alterations in the cells expressing these markers are examined through time.

A novel clone generation and marking system (MARCM, (Lee & Luo (1999)) is used to generate clones of kelch mutant cells, marked with GFP expression, in an otherwise wild type background. This allows one to focus on small groups of kelch mutant cells for changes in phenotype without the background of the whole CNS, and to determine the extent to which nearby wild type tissue may rescue the phenotypes of ketch mutant cells.

The experiments described in Examples I and II document the behavioral abnormalities associated with kelch mutations and the portions of the nervous system altered by kelch mutations, but they do not address the question of when and where Kelch is expressed. Previous results (Robinson & Cooley (1997) Development) show that Kelch protein is expressed in many, but not all, larval tissues and that there is Kelch expression in non-ovarian tissues of adult females. These results define neither the specificity of somatic Kelch expression nor the time course of expression in any particular tissue.

In order to determine if and when Kelch may be expressed in neurons, glia or other tissues, a mixture of RNA-based and protein-based procedures are used to determine the temporal and spatial pattern of kelch gene expression employing kelch DNA and monoclonal antibodies to Kelch protein (Lynn Cooley), as well as a hybridoma cell line to make and affinity purify further antibodies against Kelch.

kelch RNA is evaluated to determine if it is expressed in the CNS of larvae or in adult heads. Late third instar larvae are manually dissected and the CNS separated from larval body tissues and from the imaginal discs, which give rise to the adult cuticle. Adult male and female heads are mass separated from bodies by freezing in liquid nitrogen and rapid shaking. Heads are then purified by sieving and hand selection. RNAs are isolated from the CNS tissue and from male and female heads, and subjected to Reverse Transcriptase PCR (RT-PCR) with nested kelch primers, as we have done for dsf (Finley (1998)).

General or limited expression of kelch to a subset of cells or cell types is assayed, as well as throughout cells or in limited regions, such as axons. Dissected CNS preparations are used for whole mount antibody staining using anti-Kelch monoclonal antibodies, as has previously been done with antibodies directed at Dsf, a relatively rare protein expressed in a subset of neurons (Finley (1998)). Similarly, anti-Kelch antibodies for immunostaining of sections of adult heads and bodies to look for tissue and cell type specificity and subcellular localization are employed.

kelch and its function in preventing neural degeneration is evaluated. This includes a characterization of multiple behavioral phenotypes and correlation of these phenotypes to alteration in protein structure; characterization of the changes in CNS and other neuronal tissues in kelch mutants as animals age; and characterization of the tissues and times at which Kelch is expressed relative to the cellular and behavioral phenotypes. The cellular, biochemical and molecular role of Kelch is determined, in the context of whole organisms and behavior is built upon these studies.

The ability to add or subtract kelch function from cells or groups of cells is necessary in order to evaluate whether kelch functions in a cell autonomous manner (i.e., does the presence or absence of wild type kelch in a cell determine its phenotype?), to determine if Kelch protein is continuously needed in cells, if later addition of Kelch can prevent neural or behavioral phenotypes and if overexpression of Kelch can also generate neural or behavioral phenotypes.

To generate clones of cells that lack kelch function, the MARCM system is employed (Lee & Luo (1999)). This system is set up such that mitotic recombination using the FLP-FRT system simultaneously generates clones of cells that lack both the gene of interest, in this case wild type kelch, and the gene for the repressor protein GAL80. In the absence of GAL80, a GAL4-inducible promoter included in the stock is activated to transcribe the gene for a membrane bound GFP. This labels the cell bodies and projections of all mutant cells while leaving the surrounding wild type cells unlabeled. The appropriate kelch FRT line and stocks to take advantage of the MARCM system are employed.

Kelch protein is also expressed in wild type or mutant backgrounds in selected cells or at selected times. Different sets of cells or different times are targeted by use of the multicomponent GAL4-UAS system (Brand & Perrimon (1993)). For the purposes of this disclosure, the key goal is the construction of artificial genes in which kelch is placed under control of a GAL4-responsive promoter, and the transformation of such a gene into Drosophila.

Human proteins with substantial similarity to Kelch, including MAYVEN which is expressed notably in the brain and nervous system are also evaluated (Soltysik-Espanola, et al. (1999)). It has been observed that human proteins can substitute for Drosophila proteins, even in the nervous system (for example, for the amyloid precursor protein (Luo et al. (1992)). UAS-MAYVEN are constructed and transformed into Drosophila for rescue experiments, similar to the UAS-Kelch experiments.

Kelch is an actin binding protein with the potential for self association through N-terminal BTB domains. As such, it is believed to be actively involved in key processes in axons, dendrites and glia, in both development and neural maintenance. Indeed, studies using MAYVEN antibodies and cultured cells are consistent with the activities of Kelch identified herein (Soltysik-Espanola, et al. (1999)). The position of Kelch within cells, in cell culture, in organ culture and in organisms, at various times and under various conditions and states of neural activity are evaluated, using a visually expressed Kelch in living cells. GFP-Kelch are constructed and placed under the control of a GAL4-responsive promoter in Drosophila. Such a GFP-MAYVEN has already been shown to have at least some similar activities to those of MAYVEN alone (Soltysik-Espanola, et al. (1999)). UAS-GFP-MAYVEN can also be constructed, for example, using the existing GFP-MAYVEN or an alternate construction as can readily be accomplished by those of skill in the art.

The invention has been described in detail with reference to certain preferred embodiments thereof, however, it is recognized by those of skill in the art that variations and modifications are also within the scope of the invention.

References, Each Herein Incorporated by Reference

1. Brand, A. H. and Perrimon, N. (1993) Targeted gene expression as a means of altering cell

fates and generating dominant phenotypes. Development. 11, 401-415.

2. Buchanan, R. L. and Benzer, S. (1993) Defective glia in the Drosophila brain degeneration mutant drop-dead. Neuron. 10, 839-850.

3. Cruts, M. and Van Broeckhoven, C. (1998) Molecular genetics of Alzheimer's disease. Ann Med. 30, 560-565.

4. Ferveur, J. -F., Stortkuhl, K. F., Stocker, R. and Greenspan, R. J. (1995) Genetic feminization of brain structures and changed sexual orientation in male Drosophila. Science. 267, 902-905.

5. Finley, K. D., Edeen, P. T., Foss, M., Gross, E., Ghbeish, N., Palmer, R. H., Taylor, B. J. and McKeown, M. (1998) dissatisfaction encodes a Tailless-like nuclear receptor expressed in a subset of CNS neurons controlling Drosophila sexual behavior. Neuron. 27, 1363-1374.

6. Finley, K. D., Taylor, B. J., Milstein, M. and McKeown, M. (1997) dissatisfaction, a gene involved in sex-specific behavior and neural development of Drosophila melanogaster. Proc. Natl. Acad. Sci. USA. 94, 913-913.

7. Halter, D. A., Urban, J., Rickert, C., Ner, S. S., Ito, K., Travers, A. A. and Technau, G. M. (1995) The homeobox gene repo is required for the differentiation and maintenance of glia function in the embryonic nervous system of Drosophila melanogaster. Development. 121, 317-32.

8. Hay, B. A., Wolff, T. and Rubin, G. M. (1994) Expression of baculovirus P35 prevents cell death in Drosophila. Development. 120, 2121-2129.

9. Jackson, G. R., Salecker, I., Dong, X., Yao, X., Arnheim, N., Faber, P. W., MacDonald, M. E. and Zipursky, S. L. (1998) Polyglutamine-expanded human huntingtin transgenes induce degeneration of Drosophila photoreceptor neurons. Neuron. 21, 633-642.

10. Kretzschmar, D., Hasan, G., Sharma, S., Heisenberg, M. and Benzer, S. (1997) The swiss cheese mutant causes glial hyperwrapping and brain degeneration in Drosophila. J Neurosci. 17, 7425-7432.

11. Lee, T. and Luo, L. (1999) Mosaic analysis with a repressible neurotechnique cell marker for studies of gene function in neuronal morphogenesis. Neuron. 22, 451-461.

12. Luo, L., Tully, T. and White, K. (1992) Human amyloid precursor protein ameliorates behavioral deficit of flies deleted for Appl gene. Neuron. 9, 595-605.

13. Lush, M. J., Li, Y., Read, D. J., Willis, A. C. and Glynn, P. (1998) Neuropathy target esterase and a homologous Drosophila neurodegeneration-associated mutant protein contain a novel domain conserved from bacteria to man. Biochem J. 332, 1-4.

14. Madhani, H. D., Styles, C. A. and Fink, G. R. (1997) MAP kinases with distinct inhibitory functions impart signaling specificity during yeast differentiation. Cell. 91, 673-684.

15. Min, K. T. and Benzer, S. (1997) Spongecake and eggroll: two hereditary diseases in Drosophila resemble patterns of human brain degeneration. Curr Biol. 7, 885-888.

16. Min, K. T. and Benzer, S. (1999) Preventing neurodegeneration in the Drosophila mutant bubblegum. Science. 284, 1985-1988.

17. Mutsuddi, M. and Nambu, J. R. (1998) Neural disease: Drosophila degenerates for a good cause. Curr Biol. 8, R-809-R811.

18. O'Dell, K. M. C., Armstrong, J. D., Yang, M. Y. and Kaiser, K. (1995) Functional dissection of the Drosophila mushroom bodies by selective feminization of genetically defined subcompartments. Neuron. 15, 55-61.

19. Restifo, L. L. and White, K. (1991) Mutations in a steroid hormone-regulated gene disrupt the metamorphosis of the central nervous system in Drosophila. Dev Biol. 148, 174-94.

20. Robinson, D. N., Cant, K. and Cooley, L. (1994) Morphogenesis of Drosophila ovarian ring canals. Development. 120, 2015-25.

21. Robinson, D. N. and Cooley, L. (1997) Drosophila kelch is an oligomeric ring canal actin organizer. J Cell Biol. 138, 799-810.

22. Robinson, D. N. and Cooley, L. (1997) Examination of the function of two kelch proteins generated by stop codon suppression. Development. 124, 1405-17.

23. Rogina, B., Benzer, S. and Helfand, S. L. (1997) Drosophila drop-dead mutations accelerate the time course of age-related markers. Proc Natl Acad Sci USA. 94, 6303-6306.

24. Schüpbach, T. and Wieschaus, E. (1991) Female sterile mutations on the second chromosome of Drosophila melanogaster. II. Mutations blocking oogenesis or altering egg morphology. Genetics. 129, 1119-1136.

25. Soltysik-Espanola, M., Rogers, R. A., Jiang, S., Kim, T. A., Gaedigk, R., White, R. A., Avraham, H. and Avraham, S. (1999) Characterization of Mayven, a novel actin-binding protein predominantly expressed in brain. Mol Biol Cell. 10, 2361-2375.

26. Tatemichi, T. K., Sacktor, N. and Mayeux, R. (1994) “Dementia associated with cerbrovascular disease, other degenerative diseases, and metabolic disorders.” Alzheimer Disease. Terry, Katzman and Bick ed. Raven Press. New York. 123-166.

27. Warrick, J. M., Paulson, H. L., Gray-Board, G. L., Bui, Q., Fischbeck, K. H., Pittman, R. N. and Bonini, N. M. (1998) Expanded polyglutamine protein forms nuclear inclusions and causes neural degeneration in Drosophila. Cell. 93, 939-949.

28. Xiong, W. C. and Montell, C. (1995) Defective glia induce neuronal apoptosis in the repo visual system of Drosophila. Neuron. 14, 581-90.

29. Xiong, W. C., Okano, H., Patel, N. H., Blendy, J. A. and Montell, C. (1994) repo encodes a glial-specific homeo domain protein required in the Drosophila nervous system. Genes Dev. 8, 981-94.

30. Yang, M. Y., Armstrong, J. D., Villinsky, I., Strausfeld, N. J. and Kaiser, K. (1995) Subdivision of the mushroom bodies by enhancer-trap expression patterns. Neuron. 15, 45-54.

31. Li, M. G., Serr, M., Edwards, K., Ludmann, S., Yamamoto, D., Tilney, L. G., Field, C. M. and Hays, T. S. (1999). Filamin is required for ring canal assembly and actin organization during Drosophila oogenesis. J Cell Biol 146, 1061-74.

32. Robinson, D. N., Cant, K. and Cooley, L. (1994). Morphogenesis of Drosophila ovarian ring canals. Development 120, 2015-25.

33. Robinson, D. N. and Cooley, L. (1997). Genetic analysis of the actin cytoskeleton in the Drosophila ovary. Annu Rev Cell Dev Biol 13, 147-70.

34. Schupbach, T. and Wieschaus, E. (1991). Female sterile mutations on the second chromosome of Drosophila melanogaster. II. Mutations blocking oogenesis or altering egg morphology. Genetics 129, 1119-1136.

35. Sokol, N. S. and Cooley, L. (1999). Drosophila filamin encoded by the cheerio locus is a component of ovarian ring canals. Curr Biol 9, 1221-30.

36. Tilney, L. G., Tilney, M. S. and Guild, G. M. (1996). Formation of actin filament bundles in the ring canals of developing Drosophila follicles. J Cell Biol 133, 61-74.

That which is claimed is:

1. A method for identifying compounds that modulate Kelch activity or expression of a polynucleotide encoding Kelch, said method comprising incubating components comprising a test compound, and a Kelch polypeptide (or functional fragment thereof), or a cell expressing a Kelch polypeptide (or functional fragment thereof), under conditions sufficient to allow the components to interact, and detecting an effect of the test compound on Kelch polypeptide activity or expression of a polynucleotide encoding kelch.

2. A method for identifying proteins and other compounds which bind to, or otherwise directly interact with, Kelch or kelch, said method comprising contacting test compounds with Kelch or kelch and assaying for binding thereto.

3. A transgenic animal having a transgene encoding a member of the Kelch family or functional fragment thereof.

4. A transgenic animal according to claim 3 wherein a normal human kelch (mayven) gene has been recombinantly introduced into the genome of the animal as an additional gene, under the regulation of either an exogenous or an endogenous promoter element

5. A transgenic animal according to claim 3 wherein a mutant human kelch gene has been recombinantly introduced into the genome of the animal as an additional gene, under the regulation of either an exogenous or an endogenous promoter element.

6. A transgenic animal according to claim 3 wherein a mutant version of one of that animal's kelch genes has been recombinantly introduced into the genome of the animal as an additional gene, under the regulation of either an exogenous or an endogenous promoter element.

7. A transgenic animal according to claim 3 wherein said animal is a “Knock-out” animal in which one or both copies of one of the animal's kelch genes have been partially or completely deleted by homologous recombination or gene targeting, or have been inactivated by the insertion or substitution by homologous recombination or gene targeting of exogenous sequences.

8. A method for producing transgenic animals having a transgene encoding a member of the Kelch family or functional fragment thereof, said methods comprising introducing into the genome of a host animal a polynucleotide encoding Kelch polypeptide (or functional fragment thereof) operatively linked to a promoter which functions in said host cells to cause the production of an RNA sequence, and obtaining a transgenic animal having a nucleic acid encoding Kelch (or functional fragment thereof).

9. A transgenic animal having a transgene disrupting expression of Kelch, chromosomally integrated into the cells of said animal.

10. A transgenic animal according to claim 9 wherein said animal is a model for neurodegenerative disease associated with mutations in the kelch genes.

11. An agricultural composition comprising a mutant kelch polypeptide, a polynucleotide encoding a mutant kelch polypeptide, an antibody to a kelch polypeptide, a transgenic insect transformed with a mutant kelch transgene, and/or a compound that modulates Kelch activity or expression of a polynucleotide encoding Kelch.

12. An insecticidal composition comprising a transgenic insect having disrupted expression of Kelch and/or one or more compounds that modulate Kelch activity or expression of a polynucleotide encoding Kelch.

13. An insecticidal composition according to claim 12 wherein said composition comprises a transgenic insect carrying a transgene comprising DNA disrupting expression of Kelch and an agriculturally acceptable carrier.

14. An insecticidal composition according to claim 13 wherein said transgenic insect carries a transgene comprising DNA disrupting expression of a nucleic acid encoding Kelch, in a manner such that these polynucleotides are stably integrated into the DNA of germ line cells of the mature insect and inherited in normal Mendelian fashion.

15. An insecticidal composition according to claim 12 comprising a compound which modulates Kelch biological activity or expression of a Kelch polypeptide.

16. A method of screening for carriers of Kelch alleles associated with age-associated neurodegenerative diseases, said method comprising assaying for the presence of proteins, antibodies, polynucleotides and/or activities indicative of abnormal Kelch sequences.

17. A method according to claim 16 wherein said neurodegenerative disease includes dementias, psychiatric disease, decreased sexual ability and desire, decreased motor skills and other neurologic diseases, disorders and behaviors associated with age.

18. A pharmaceutical preparation for use in the treatment of Kelch-associated diseases, said preparation comprising a normal Kelch protein, a normal Kelch gene, an antisense sequence to mutant Kelch genes or which “knock-out” the mutant genes, sequences which encode a protein which blocks or corrects the deleterious effects of Kelch mutants, antibodies to normal and/or mutant Kelch proteins, or small molecules (drugs) which alter Kelch expression, block or enhance abnormal interactions between mutant forms of Kelch and other proteins or ligands, or which otherwise block or enhance the aberrant function of mutant Kelch proteins by altering the structure of the mutant proteins, by enhancing their metabolic clearance, or by inhibiting their function, or by enhancing the decreased interaction of mutant forms of Kelch.

19. A method for the treatment of Kelch-associated diseases, said method comprising administering normal Kelch proteins, administering normal Kelch genes to compensate for or replace the mutant genes, administering antisense sequences to mutant Kelch genes or which “knock-out” the mutant genes, administering sequences which encode a protein which blocks or corrects the deleterious effects of Kelch mutants, administering antibodies to normal and/or mutant Kelch proteins, or administering small molecules (drugs) which alter Kelch expression, block or enhance abnormal interactions between mutant forms of Kelch and other proteins or ligands, or which otherwise block or enhance the aberrant function of mutant Kelch proteins by altering the structure of the mutant proteins, by enhancing their metabolic clearance, or by inhibiting their function, or by enhancing the decreased interaction of mutant forms of Kelch.
